(19) United States
(12) Patent Application Publication
(10) Pub. No.: US 2026/0315102 A1
(43) Pub. Date: Oct. 15, 2026

(54) SPINAL NEEDLE WITH CEREBROSPINAL FLUID SENSOR

(71) Applicant: Armando De Jesus VELEZ ROJAS, JALISCO (MX)

(72) Inventor: Armando De Jesus VELEZ ROJAS, JALISCO (MX)

(21) Appl. No.: 19/106,517

(22) PCT Filed: Aug. 29, 2022
(86) PCT No.: PCT/IB2022/058087

§ 371 Date: Feb. 25, 2025

(51) Int. Cl. A61B 17/34

(52) U.S. Cl. CPC A61B 17/3401; A61B 17/3421; A61B 17/3494

(57) ABSTRACT

A spinal needle with cerebrospinal fluid (CSF) detector is described, wherein the needle of the present invention comprises a hub comprising a proximal end and a distal end, a CSF detector, a cannula comprising a tip, which is fluidically attached to the distal end of said hub; wherein the hub additionally comprises, at its proximal end, a port for connecting a device for medical fluids, and a protruding ring configured with a first tactile mark formed on its upper external part and extending longitudinally thereon, which is oriented in the same direction with respect to the of the cannula.

### FIELD OF THE INVENTION

The present invention relates to the design of devices and equipment for surgical procedures, and more particularly, it relates to a spinal needle with cerebrospinal fluid (CSF) detector.

### BACKGROUND OF THE INVENTION

There are two basic techniques for administering medications into a patient's spinal area: epidural and subdural, these can be used to create spinal anesthesia or analgesia. In this type of surgical procedure, an anesthetic is injected into the spinal cord and nerve roots in order to block pain sensations from a region of the body, such as the abdomen, hips, legs, or pelvis during childbirth.

These medical techniques involve some risks for patients, for this reason, it is important to consider the main parts of the human body that participate in these procedures, such as the meninges and cerebrospinal fluid that protect the central nervous system. As is well known, the meninges are composed of three layers: the dura mater, the arachnoid mater, and the pia mater. The dura mater is the strongest, most inflexible, and outermost of the three layers; the arachnoid mater is the middle membrane, and the pia mater is the innermost and most delicate of the meningeal layers. While cerebrospinal fluid is a clear, colorless body fluid with a water-like consistency that occupies the subarachnoid space, which is the space between the arachnoid layers and the pia mater of the meninges.

Epidural anesthesia involves the insertion of a hollow spinal needle (for example, a Tuohy needle), as well as the introduction of a flexible catheter into the space between the spine and the outer membrane of the spinal cord (epidural space) in the middle or lower back. The area where the needle is inserted is blocked with local anesthesia. The needle is then inserted and removed after the catheter has passed through the epidural space, leaving the catheter in this area. Anesthetic is injected into the catheter to block the area of the body above or below the injection site, as needed. The catheter is secured on the back so that it can be used again if more anesthetic is needed.

Subdural or spinal anesthesia is performed in the same manner, except that the anesthetic is injected directly into the cerebrospinal fluid surrounding the spinal cord with the aid of a second spinal needle, such as a Whitacre needle, a Quincke needle, or a Sprotte needle, which is then inserted into the first spinal needle (Tuohy needle). Subdural anesthesia blocks the part of the body below the site where the anesthetic is administered, depending on the dose of the anesthetic and the technique used to apply it.

Sometimes a spinal catheter may be inserted and left in place at the injection site to provide continuous spinal anesthesia instead of using the second needle.

Any spinal needle must pierce the skin, subcutaneous fat, supraspinous ligament, ligaments, epidural space (in the case of epidural anesthesia), dura mater, and arachnoid layer until the needle reaches the subarachnoid space, which contains the spinal cord and nerve roots that are surrounded by CSF (in the case of subdural anesthesia).

On the other hand, there is also mixed anesthesia, which consists of the application of a combination of two anesthetic techniques, such as the combination of epidural and subdural anesthesia. In mixed anesthesia, balanced anesthesia can be performed by first dosing a dose to the subdural space, this dose will have the anesthetic effect for the entire surgery, and subsequently, if necessary, supplying small booster doses through the epidural catheter to the epidural space itself, this balanced technique avoids the use of large volumes of anesthetic, in addition to minimizing the toxicity of anesthetics to the patient during the procedure.

In epidural or subdural anesthesia, it is particularly important to ensure that the injection reaches the desired area correctly; otherwise, the anesthetic can affect the nervous, cardiovascular, and respiratory systems. Both epidural and subdural anesthesia can significantly affect breathing, heartbeat, and other vital functions. In addition, there is a potential risk of toxicity caused by large doses of medication not needed to obtain adequate blockade. As mentioned, the flow direction in which the anesthetic is dosed into the epidural or subarachnoid space is particularly important to achieve the goal of blocking the desired body part.

In an effort to make improvements to methods and devices for localizing the epidural or subarachnoid space, several proposals have been developed as disclosed in the state of the art below:

U.S. Pat. No. 6,925,323, which relates to a system for increasing visibility in the epidural space, discloses a method of epidural surgery that improves visibility in a patient's epidural space for the purpose of effectively conducting therapeutic surgery in said space. The method includes the steps of distending a portion of the patient's epidural space by filling the portion of the epidural space with a fluid administered from a catheter; placing an optical sight glass into the distended portion of the epidural space, inserting the optical sight glass through the same catheter that supplies the distending fluid, thereby providing a visual image of the epidural space.

U.S. Pat. No. 6,773,417 shows an epidural space locator device comprising a body section having a first end and a second end; a channel extending between said ends, wherein the first end is attachable to a Luer connector; and, a collapsible posterior chamber having one end attached to the second end of the body section and the other end being exposed in such a manner as to allow pressure to be exerted with one or more fingers of the hand, such that when there is a positive pressure within the chamber, it maintains its shape and when the pressure is negative or zero within the chamber, it collapses, indicating the location of the epidural space through a needle that attaches to a Luer connector; and the loss of pressure within the chamber is detected with the fingers as the shape of the chamber collapses, thus requiring highly trained personnel to detect the change in pressure.

U.S. Pat. No. 6,558,353 discloses a needle comprising a needle hub disposed at the proximal end of a hollow needle. The needle hub has port indicators that provide visual and tactile verification to a user regarding the orientation of the needle tip, and more specifically, the needle hub includes projections for tactile verification and a magnifying window for visual verification. However, both the projections and the window are very small and visual detection of CSF through the needle is not always achieved. In fact, sometimes small air bubbles accumulate under the window, distorting the view when a user looks through it.

Additionally, the Whitacre and Tuohy needles, known in the prior art, include a small groove that helps the surgeon know the orientation of the needle tip; however, in this type of simple needles, as well as in the needle of U.S. Pat. No. 6,925,323, it has not been possible to effectively detect the presence of CSF through the needle in a visual manner. The magnifying window of U.S. Pat. No. 6,925,323 is very small compared to the hub of the needle. A common medical practice to detect CSF has been to drip CSF into the proximal end of the spinal needle, however, if there is excessive loss of CSF, the patient often suffers from headaches. For this reason, it is necessary to clearly detect the presence of CSF in order to dose the medication or anesthetic in the correct direction and location of the meninges, otherwise, if CSF cannot be obtained through the needle, the surgical process has to be delayed or abandoned.

Mexican patent application MX/a/2007/006751 describes a needle for determining the direction of the needle tip once the cerebrospinal fluid is inside the needle in a chamber incorporated therein; however, because the cerebrospinal fluid is clear and almost transparent, optical identification is difficult, repeating many of the problems mentioned above.

For all the above, although there are some devices and methods to locate the epidural or subarachnoid space, there is still the inconvenience of visually detecting the CSF through spinal needles, so efforts has been made to eliminate the inconveniences presented by the spinal needles used today, thus developing a spinal needle with a CSF detector that, in addition to including a thermochromic element that changes color and indicates the presence of CSF in the needle, does not involve excessive loss of CSF in the patient.

### Objects of the Invention

Considering the shortcomings of the prior art, it is an object of the present invention to provide a spinal needle with a CSF detector that allows identifying the presence of CSF in order to dose the medication or anesthetic in the correct direction and place of the meninges and that does not involve excessive loss of CSF in the patient.

These and other objects are achieved by a spinal needle with CSF detector in accordance with the present invention.

### BRIEF DESCRIPTION OF THE INVENTION

To overcome the problems of the prior art with respect to the correct detection and visualization of CSF in conjunction with the orientation of the tip of spinal needles, a spinal needle has been developed, characterized in that it comprises: a hub comprising a proximal end, a distal end and a CSF detector; and, a cannula attached to the distal end of said hub that is in flow communication with said hub.

Another aspect of the invention considers a spinal needle further characterized in that the CSF detector can be in a housing or in a reservoir protruding from said hub.

### BRIEF DESCRIPTION OF THE FIGURES

The novel aspects which are considered characteristic of the present invention will be set forth with particularity in the appended claims. However, the invention itself, both as to its structure and operation, together with other objects and advantages thereof, will be better understood from the following detailed description, when read in conjunction with the accompanying drawings, in which:

FIG. 1 is an isometric view of a first embodiment of the spinal needle with CSF detector.

FIG. 2 is an exploded view of the spinal needle in a first embodiment.

FIG. 3 is an exploded view of the spinal needle hub in a first embodiment.

FIG. 4 is an isometric view of a spinal needle with a CSF detecting reservoir constructed in accordance with a second embodiment of the present invention.

FIG. 5 is an exploded view of the needle of FIG. 4 including a protector for the needle cannula.

FIG. 6 is a perspective view of the hub area of the needle of FIG. 4 and without the stylet cap thereof.

FIG. 7 is a perspective view of the hub area of the needle of FIG. 4 with stylet cap.

FIG. 8 is a right-side view of the hub area of the needle without a stylet cap shown in FIG. 4.

FIG. 9 is a side sectional view of FIG. 8.

FIG. 10 is a top plan view showing the coupling between the needle of FIG. 4 and a Tuohy type needle.

### DETAILED DESCRIPTION OF THE INVENTION

It has been found that a spinal needle capable of facilitating the detection of CSF in the needle is required to dose the medication or anesthetic in the correct direction and place of the meninges and that does not involve excessive loss of CSF in the patient, so the present invention relates to a spinal needle characterized in that it comprises: a hub comprising a proximal end, a distal end and a CSF detector; and, a cannula attached to the distal end of said hub that is in flow communication with said hub.

For explanatory purposes and in accordance with medical practice, the distal direction is that which is closest to the patient, while the proximal direction is that which is towards the medical staff.

Said hub has at its proximal end a port to connect a device for medical fluids.

In one embodiment of the invention, the port is a Luer type connector.

The hub has a first tactile mark formed on its external surface to know the orientation of the tip of the cannula, which is oriented in the same direction with respect to the first tactile mark.

In a preferred embodiment of the invention, the first tactile mark is a groove, wherein said groove is ergonomically designed to be manipulated by the user's index finger.

In another preferred embodiment of the invention, the hub has a second tactile mark oriented in the same direction with respect to the tip of the cannula.

In one embodiment of the invention, the hub has a transparent housing.

In one embodiment of the invention, the housing is cylindrical in shape with rectangular projections protruding from the periphery of the cylinder to facilitate manipulation by the user.

In one embodiment of the invention, the CSF detector is within the housing.

In one embodiment of the invention, the CSF detector is a thermochromic element that changes color when it comes into contact with CSF.

In one embodiment of the invention, the CSF detector within the housing is located on the same longitudinal axis as the cannula.

In another embodiment of the invention, the CSF detector is within a reservoir that protrudes from the hub, on an axis parallel to the longitudinal axis of the cannula. Likewise, said reservoir has right and left walls, a proximal wall, a distal wall, a bottom wall, and an open top wall where the CSF is received from the cannula to fall to the bottom of the reservoir and fill it to cover the thermochromic element.

In a preferred embodiment of the invention, the reservoir that receives the CSF is transparent.

In a preferred embodiment of the invention, the reservoir contains the thermochromic element, which changes color when the CSF is in said reservoir.

The reservoir is located at the distal end of the hub, next to the cannula, to avoid using an unnecessary amount of CSF for identification.

In a preferred embodiment of the invention, the reservoir has the specific and necessary capacity and dimensions to avoid collecting an unnecessary excess volume or having any cerebrospinal fluid leak that puts the patient at risk.

In a preferred embodiment of the invention, the reservoir has a capacity of 0.001 milliliters to 10 milliliters.

In a preferred embodiment of the invention, the bottom wall is flat to support the needle on a surface.

The reservoir is designed in such a way that a user can place his fingers on the left wall and the right wall and thus take the needle.

In a preferred embodiment of the invention, the proximal wall includes a recess in which a portion of the thermochromic element is received.

Regarding the thermochromic element, it is made of a plastic material with dye, which reacts when detecting the temperature of the CSF. The thermochromic element is fixed inside the reservoir, although it is possible that it is floating. The thermochromic element changes from one base color to a color different from the base color when the CSF is in the reservoir. More particularly, the thermochromic element begins to change color from 30 °C, which is appropriate because the temperature of the operating rooms is around 25 °C and remains constant, in this way the thermochromic element only changes its color due to the presence of CSF and not due to ambient temperature.

In one embodiment of the invention, the thermochromic element is located between the bottom and the top wall, running through the space between the proximal wall and the distal wall to ensure contact of the cerebrospinal fluid with the thermochromic element and thus make the correct visual detection of the presence of cerebrospinal fluid in the needle.

In a preferred embodiment of the invention, the thermochromic element has a cylindrical shape.

Finally, said cannula has a tip selected from the group consisting of a Whitacre pencil tip, a Quincke tip or a Sprotte tip.

The combination of these elements forms a spinal needle with a thermochromic element that allows detecting the presence of CSF through the needle during anesthesia and analgesia procedures, among other surgical operations.

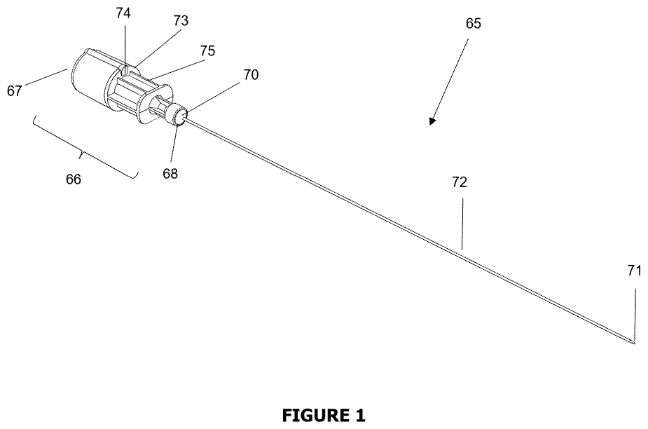

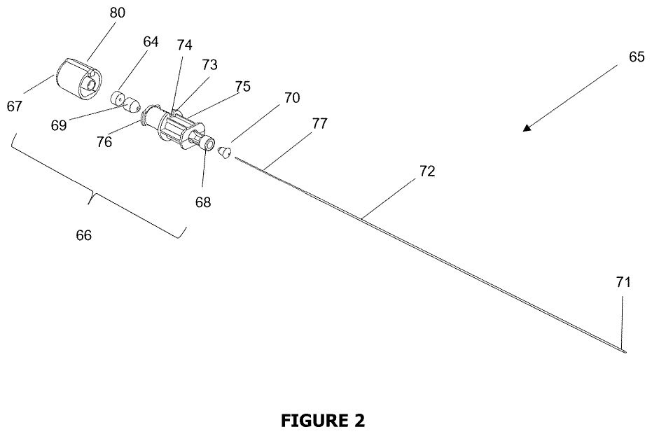

Referring now to FIGS. 1 and 2, these illustrate a first embodiment of the invention comprising a spinal needle (65) which is integrated from a hub (66) with a proximal end (67) and a distal end (68), wherein said hub (66) contains a housing (75) which is made of a transparent material that facilitates observing the interior thereof; a CSF detector (69) which is inserted and housed on the internal part of said housing (75). Likewise, the spinal needle (65) comprises a circular introducer hub (70) with a middle perforation, which allows the coupling of said detector (69) within the housing (75).

Additionally, and in order to avoid the introduction of any foreign material into the spinal needle (65) before its use, a stylet (77) is optionally used within a cannula (72), wherein said stylet (77) is attached to the inner part of a stylet cap (80), arranged to be jointly coupled to the proximal end (67) of the hub (66). In a preferred embodiment of the invention, the hub also contains an insert (64) with a central bore, which serves to center the stylet (77) when introducing it into the cannula (72).

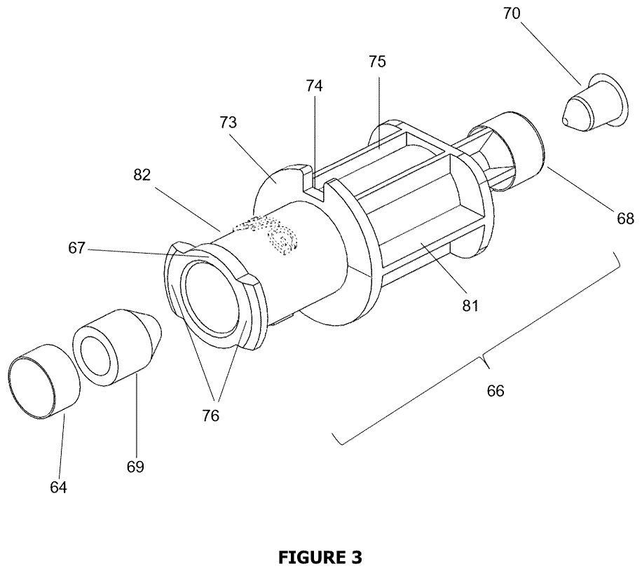

In accordance with what is shown in FIG. 3, in a further aspect of the first embodiment of the invention, the hub (66) integrally includes a protruding ring (73) configured with a first tactile mark (74) in the form of a groove formed and located at one end of said protruding ring (73), wherein said first tactile mark (74) is ergonomically designed to be manipulated by at least one finger of a user's hand and thus allow the orientation of the tip (71) of the cannula (72) to be known and thus be able to properly direct the tip (71) of the cannula (72). Furthermore, the housing (75) has a special ergonomic configuration in a cylindrical shape with rectangular projections (81) protruding on the periphery of the cylinder to facilitate manipulation by the user. The housing (75) comprises a transition section (82) comprising a pair of clamping guides (76) that serve to guide the coupling of the stylet cap (80).

With respect to the CSF detector (69), it is inserted and housed by the internal part of said housing (75), said detector is preferably configured in a cylindrical shape with a conical tip, but it can have some other geometry as long as it is in contact with the CSF. Likewise, the spinal needle (65) comprises a circular introducer hub (70) with a middle perforation, which allows the coupling of said detector (69) within the housing (75).

In a second preferred embodiment of the invention, there is an additional spinal needle design having the same functionalities as the first embodiment, but with a different configuration.

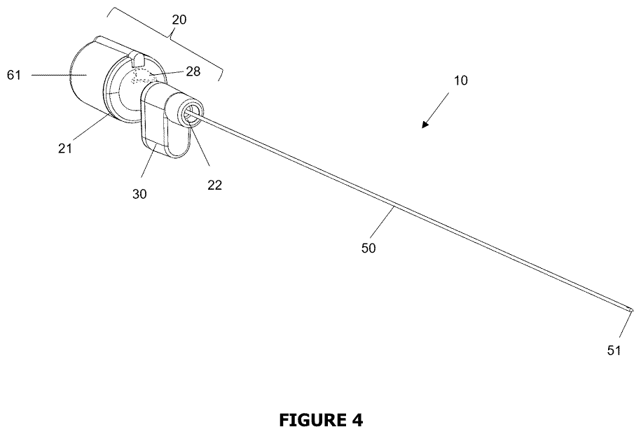

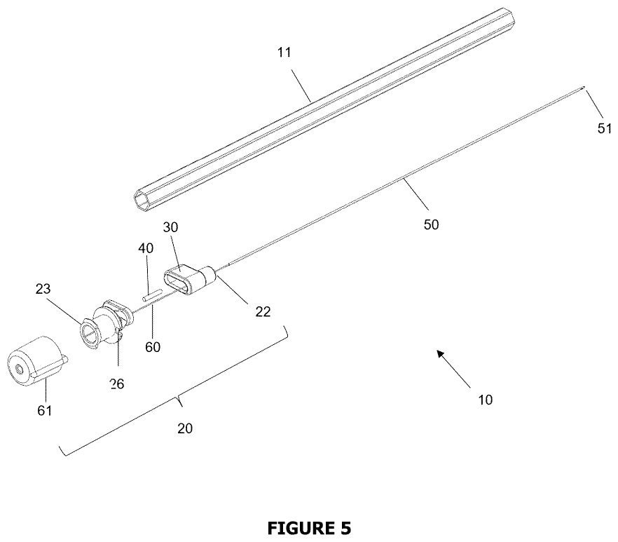

FIGS. 4 and 5 show a second embodiment of a spinal needle (10) with a reservoir (30) containing a CSF detector (40).

From what is shown in FIG. 4, the spinal needle (10) is integrated from the following elements: a hub (20) with a proximal end (21) and a distal end (22); wherein additionally the hub (20) comprises a reservoir (30) that is attached to the hub (20), in which said reservoir (30) for the CSF projects downwards from the hub (20), on an axis parallel to the longitudinal axis of the cannula; wherein said reservoir (30) is made of a transparent material that facilitates observing the interior thereof.

Additionally, the spinal needle (10) comprises a CSF detector (40) which is a thermochromic element housed within the reservoir (30), wherein said thermochromic element changes color when the CSF is in said reservoir (30); and, finally, there is a cannula (50) attached to the distal end (22) of said hub (20) and being in fluid communication with said reservoir (30), which is located immediately next to the distal end (22) of the hub (20) and next to the cannula (50) whereby the first quantities of CSF received in the needle are used to perform its identification.

Additionally, said reservoir (30) is configured with right and left walls, a front wall, a rear wall, a bottom wall, and an open top wall where the CSF is received to fall to the bottom of the reservoir, with the appropriate capacity and dimensions to avoid excessive loss of CSF. This capacity can be between 0.001 milliliters to 10 milliliters.

The extreme tip (51) of the cannula (50) has a pencil tip configuration. Other types of tips may be used for said cannula (50), such as the Quincke type tip and the Sprotte type tip.

In order to prevent the introduction of any foreign material into the spinal needle (10) prior to use, a stylet (60) is optionally used within the cannula (50), wherein the stylet (60) is attached to a stylet cap (61).

Similarly, and in particular in FIG. 5, a protector (11) is shown that covers the cannula (50) to avoid any incidents since it serves as protection when the spinal needle (10) is not used.

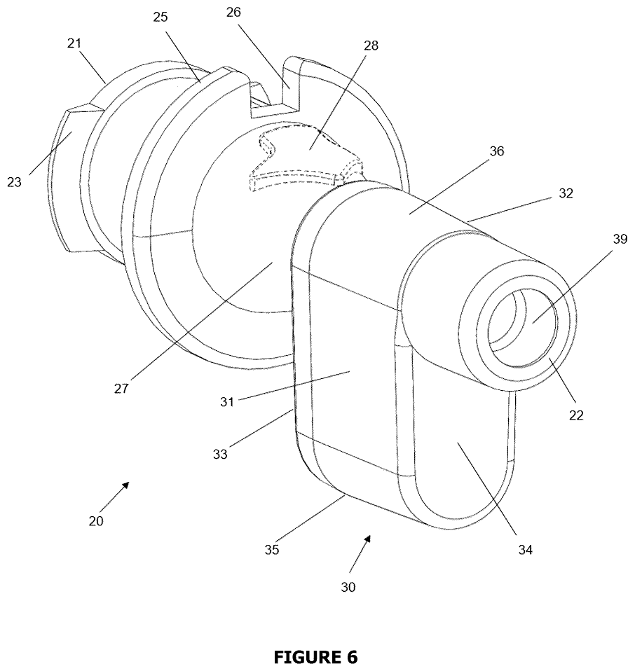

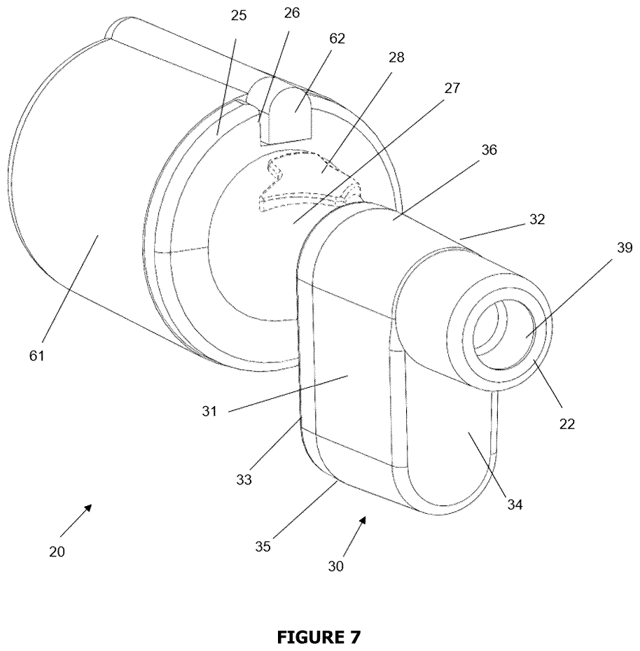

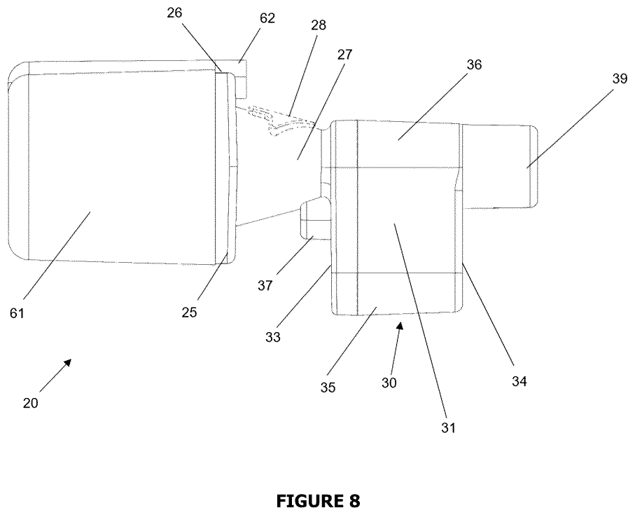

Now, FIGS. 6, 7 and 8 are enlarged views of the hub (20) of FIG. 4 without showing the entire cannula.

In a further aspect of the invention, the hub (20) integrally includes a protruding ring (25) that is configured with a first tactile mark (26) in the form of a groove, formed in the upper part of the protruding ring (25) and extending longitudinally thereon, wherein said first tactile mark (26) is ergonomically designed to be manipulated by the index finger of a user and thus allows to know the orientation of the tip (51) of the cannula (50) that is oriented in the same direction with respect to the first tactile mark (26) to correctly direct the tip (51) of the cannula (50).

The hub (20) is configured with a transition section (27) of conical shape and in fluid communication with the reservoir (30), where said transition section (27) includes a second tactile mark (28) that can be in the form of an arrowhead relief arranged on the upper part of said transition section (27) and with the same orientation of the tip (51) of the cannula (50).

Regarding the reservoir (30), it protrudes from the hub (20) in an axis parallel to the longitudinal axis of the cannula. As seen in FIG. 6, the reservoir (30) has a right side wall (31), a left side wall (32), a proximal wall (33), a distal wall (34), a bottom wall (35) and an upper wall (36), where the proximal wall (33) comprises an opening (39) through which the CSF is received from the cannula (50) to fall to the bottom of said reservoir (30) and fill it progressively, in addition the reservoir (30) has the capacity and dimensions necessary to avoid using an amount of CSF that puts the patient at risk.

The bottom wall (35) is configured with a curved geometry to support the spinal needle (10) on a surface. Furthermore, the reservoir (30) is designed in such a way that the user can easily manipulate/handle it by a hand, in order to hold the right wall (31) and the left wall (32) and thereby direct the spinal needle (10) and introduce it, for example, into a Tuohy-type needle. In this sense, the manipulation technique is conducted by using the middle finger and thumb to hold the spinal needle (10), while the index finger is used to locate the first tactile mark (26) and the second tactile mark (28).

Now, the present invention stands out for including the CSF detector (40) which is arranged between the bottom wall (35) and the upper wall (36) extending to the distal wall (34) and the proximal wall (33) to guarantee the contact of the CSF with the CSF detector (40) and thus the correct detection of the presence of cerebrospinal fluid. Furthermore, the proximal wall (33) includes a proximal recess (37) and the distal wall (34) includes a distal recess (38), wherein each proximal (37) and distal (38) recess receives and houses each end of the CSF detector (40). The CSF detector (40) has a configuration that allows it to be in contact with the CSF, where it is preferably a cylindrical configuration and is made of a plastic material with dye, which reacts when detecting the temperature of the CSF. Likewise, the LCR detector (40) can be arranged in a fixed manner or floating inside the reservoir (30).

The thermochromic element is configured to change from one base color to a color different from the base color when the CSF is in the reservoir (30), where the thermochromic element starts to change color from 30 °C, which is suitable because the temperature of the operating rooms is around 25 °C which remains constant, in this way, the thermochromic element only changes color due to the presence of cerebrospinal fluid and not due to the ambient temperature surrounding the needle or the manipulation of the needle by the medical staff since the walls of the reservoir prevent heat transmission.

In the present invention, the proximal end (21) includes a port (23) for connecting a medical fluid handling device, such as a syringe or catheter containing a medication or an anesthetic to be dosed. In the embodiment described, said port (23) is provided in the form of a “Luer” type connector (shown in FIG. 5); however, other types of connectors can be used for the same purpose.

In accordance with what is shown in FIG. 5, and in order to avoid the introduction of any foreign material into the needle (10) before its use, a stylet (60) is used inside the cannula (50), where said stylet (60) is attached to a stylet cap (61), which includes a tooth (62) that fits into the groove (26). In this regard, by engaging the tooth (62) within the groove (26), the stylet cap (61) envelops a portion of the proximal end of the hub (20) and the port (23) in order to protect it. With reference to the cannula (50), said cannula is attached to the distal end (22) of the hub (20) and is in flow communication with the reservoir (30), in addition said cannula (50) is very thin because it is designed to pierce the dura mater, thus reducing the patient's headache after the operation; in addition, the cannula (50) provides precise access and gives greater control for the safety and comfort of the patient. The tip (51) of the cannula (50) is preferably of the pencil tip type and has the same orientation as the first groove of the hub (20) and the second tactile mark (28).

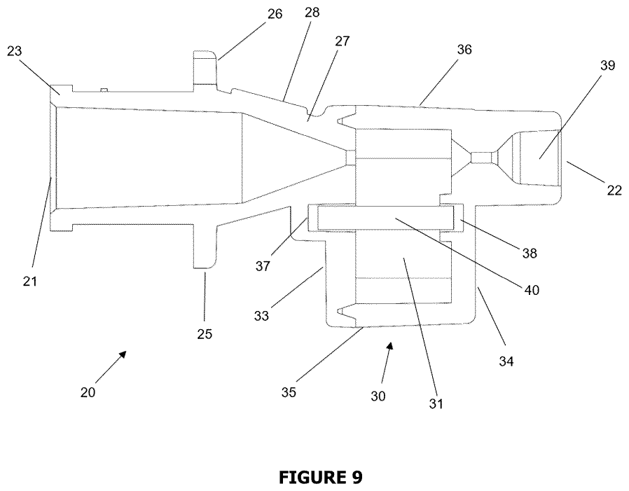

As shown in FIG. 9, the CSF flows through the spinal needle (10). When the user has located the subarachnoid space, the CSF flows through the interior of the cannula (50) towards the reservoir (30); in which, a small amount of CSF is collected covering the thermochromic element, then the CSF, having a temperature of approximately 36 °C causes the thermochromic element to change color, thus alerting the medical staff about the presence of CSF in the needle and which can be easily seen from any of the walls of the reservoir (30).

Once the user is certain that he has located the subarachnoid space with the presence of CSF in the reservoir (30), the user can attach the medical fluid management device to the port (23) at the proximal end (21) of the hub (20), and then the surgeon doses the anesthetic or medication through the spinal needle (10). The reservoir (30) does not affect the dosage of the anesthetic or the medication.

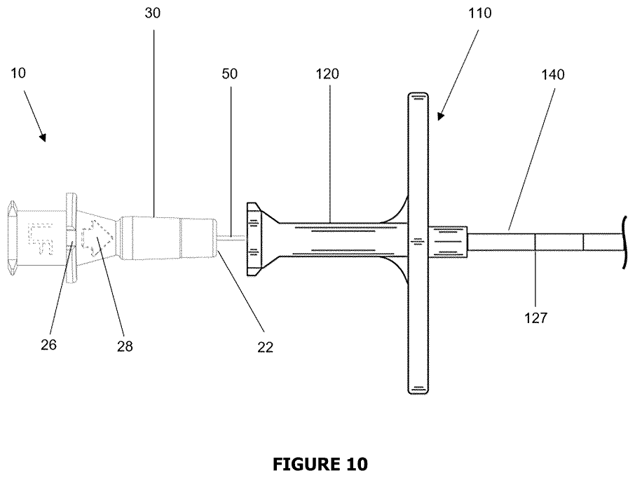

In FIG. 10, it is shown how the spinal needle (10) of the preferred embodiment of this invention is coupled to a Tuohy needle (110), which has a hub (120) and a cannula (140). The Tuohy needle (110) is used to locate the epidural space, and then the spinal needle (10) can be introduced to effectively detect the presence of CSF which can be seen through the walls of the reservoir (30) by identifying the color change of the thermochromic element.

While coupling between both needles (10) and (110), the cannula (50) of the spinal needle (10) is introduced along the cannula (140) of the Tuohy needle until the distal part (22) of the spinal needle (10) is inside the hub (120) of the Tuohy needle (110). The cannula (140) has markings (127) that indicate how deep the cannula (140) has entered the patient's tissues. The groove (26) and mark (28) allow the user to know the orientation of the tip (51) of the cannula (50) regardless of whether the tip is inside the Tuohy needle.

The Tuohy, Whitacre, Quinckle or Sprotte needles of the prior art may be configured in accordance with the principles of the present invention, i.e., the spinal needle reservoir of the present invention may be provided on such types of needles as are well known.

The combination of these elements forms a spinal needle with reservoir, which is used to detect the presence of CSF through the needle during anesthesia and analgesia procedures, among other surgical operations.

In accordance with the above, it will be seen that the spinal needle with cerebrospinal fluid detector has been designed to facilitate the detection of CSF in the needle to dose the medication or the anesthetic in the correct direction and place of the meninges without requiring an excessive amount of the patient's CSF, and it will be apparent to any person skilled in the art that the embodiments of the spinal needle with cerebrospinal fluid detector, as described above and illustrated in the accompanying drawings, are only illustrative and not limiting of the present invention, since numerous considerable changes in its details are possible without departing from the scope of the invention. For example, the reservoir may be located in a different location, as may the detector, and distinct types of CSF detectors, connectors or needles may be used depending on the requirements of the procedure to be performed.

Therefore, the present invention should not be considered as restricted except as required by the prior art and by the scope of the appended claims.

1. A spinal needle comprising:
a) a hub comprising a proximal end and a distal end;
b) a CSF detector;
c) a housing configured to house the CSF detector and receive the CSF; and
d) a cannula comprising a tip, which is fluidically attached to the distal end of said hub;
wherein the hub additionally comprises, at its proximal end a port for connecting a device for medical fluids, and
a protruding ring configured with a first tactile mark formed on its upper external part and extending longitudinally thereon, which is oriented in the same direction with respect to the tip of the cannula; and
wherein the housing has a cylindrical shape with rectangular projections protruding therefrom.

2. The spinal needle according to claim 1, wherein the port is a Luer type connector.

3. The spinal needle according to claim 1, wherein the hub additionally comprises a second tactile mark oriented in the same direction with respect to the tip of the cannula.

4. The spinal needle according to claim 1, wherein the CSF detector is a thermochromic element that comes into contact with the CSF and changes color from a temperature of 30 °C.

5. The spinal needle according to claim 1, further comprising a stylet within the cannula wherein said stylet is attached to the inner part of a stylet cap arranged to jointly engage the proximal end of the hub.

6. The spinal needle according to claim 1, wherein the cannula has a pencil tip configuration.

7. The spinal needle according to claim 6, wherein the cannula has a tip selected from the group consisting of a Whitacre pencil tip, a Quincke tip or a Sprotte tip.

8. The spinal needle according to claim 1, wherein the housing is transparent.

9. The spinal needle according to claim 1, wherein the housing is located on the same longitudinal axis as the cannula.

10. The spinal needle according to claim 1, further comprising an introducer hub with a perforation, which allows the coupling of the CSF detector within the housing.

11. A spinal needle comprising:
a) a hub comprising a proximal end and a distal end;
b) a CSF detector;
c) a reservoir protruding from the hub configured to contain the CSF detector and receive the CSF; and
d) a cannula comprising a tip which is fluidically connected to the distal end of said hub;
wherein the hub additionally comprises, at its proximal end a port for connecting a device for medical fluids, and
a protruding ring configured with a first tactile mark formed on its upper external part and extending longitudinally thereon, which is oriented in the same direction with respect to the tip of the cannula;
wherein the reservoir has a right side wall a left side wall, a proximal wall a distal wall, a bottom wall and an upper wall the proximal wall comprises an opening through which the CSF is received from the cannula to fall to the bottom of said reservoir and fill it progressively,
wherein the proximal and distal walls each comprise, a recess housing a CSF detector, so that it is disposed between the bottom wall and the upper wall of the reservoir.

12. The spinal needle according to claim 11, wherein the port is a Luer type connector.

13. The spinal needle according to claim 11, wherein the hub additionally comprises a second tactile mark oriented in the same direction with respect to the tip of the cannula.

14. The spinal needle according to claim 11, wherein the CSF detector is a thermochromic element that comes into contact with the CSF and changes color from a temperature of 30 °C.

15. The spinal needle according to claim 11, further comprising a stylet within the cannula wherein said stylet is attached to the inner part of a stylet cap arranged to jointly engage the proximal end of the hub

16. The spinal needle according to claim 11, wherein the cannula has a pencil tip configuration.

17. The spinal needle according to claim 16, wherein the cannula has a tip selected from the group consisting of a Whitacre pencil tip, a Quincke tip or a Sprotte tip.

18. The spinal needle according to claim 11, wherein the reservoir that receives the CSF is transparent.

19. The spinal needle according to claim 11, wherein the reservoir contains the CSF detector on an axis parallel to the longitudinal axis of the cannula.

20. The spinal needle according to claim 11, wherein the reservoir is located at the distal end of the hub next to the cannula.

21. The spinal needle according to claim 11, wherein the reservoir has a capacity of between 0.001 milliliters to 10 milliliters.

22. The spinal needle according to claim 11, wherein the bottom wall of the reservoir is flat for supporting the needle on a surface.

23. The spinal needle according to claim 11, wherein the CSF detector has a cylindrical shape.

24. The spinal needle according to claim 11, wherein the hub is configured with a transition section of conical shape and in fluid communication with the reservoir.
